(19) United States
(12) Patent Application Publication
(10) Pub. No.: US 2021/0228484 A1
(43) Pub. Date: Jul. 29, 2021

(54) VETERINARY THICK GEL COMPOSITION OF HIGH PALATABILITY CONTAINING ANTIOXIDANTS TO DECREASE SARCOPENIA, OSTEOPENIA AND DNA DAMAGE FROM OXIDATIVE STRESS IN DOGS, AND PROCEDURE FOR ITS OBTENTION

(71) Applicants: Daniel BIDNER, Caba (AR); Yolanda ARIAS, North Miami, FL (US)

(72) Inventor: Carlos Alberto MONTAGNA, Buenos Aires (AR)

(73) Assignees: Daniel BIDNER, Caba (AR); Yolanda ARIAS, North Miami, FL (US)

(21) Appl. No.: 17/048,966

(22) PCT Filed: Apr. 17, 2019
(86) PCT No.: PCT/US2019/027791

§ 371 Date: Oct. 19, 2020

Related U.S. Application Data
(60) Provisional application No. 62/659,885, filed on Apr. 19, 2018.

(51) Int. Cl. A61K 9/00; A61K 47/46; A61K 31/05; A61K 31/355; A61K 47/26; A61K 31/09; A61K 47/44; A61P 39/06; A61K 47/36; A61K 9/06; A23K 50/42; A23K 20/174; A23K 20/111; A23K 10/20; A23K 20/158

(52) U.S. Cl. CPC A61K 9/0056; A23K 20/158; A61K 31/05; A61K 31/355; A61K 47/26; A61K 31/09; A61K 47/44; A61P 39/06; A61K 47/36; A61K 9/06; A23K 50/42; A23K 20/174; A23K 20/111; A23K 10/20; A61K 47/46

(57) ABSTRACT

Veterinary thick gel composition of high palatability containing antioxidants to decrease sarcopenia, osteopenia and DNA damage from oxidative stress in dogs, which consists of the combination of at least two antioxidants selected from: Resveratrol, Pterostilbene, Vitamin E, Beta-Carotene, Myricetin and Quercetin at a concentration between 0.1% and 5.0% w/w, which are dissolved in a carrier of high palatability together with excipients accepted for veterinary purposes. Procedure for obtaining said veterinary composition.

### FIELD OF THE INVENTION

This invention refers to veterinary compositions, particularly to antioxidant compositions for dogs, and more particularly, it refers to compositions of improved palatability for dogs.

### BACKGROUND OF THE INVENTION

In the state of the art, there are several known compounds with antioxidant capacity. Among these antioxidant compounds, it is possible to highlight the use of different families of substances, such as Flavonols, Polyphenols, Tocopherols, Terpenes, etc.

Among the family of Flavonols we may find Myricetin, Quercitin and Kaempferol; among the Polyphenols, the Resveratrol and Pterostilbene stand out; Vitamin E belongs to Tocopherols, and Carotenes are among Terpenes.

Additionally, there are precedents of antioxidant synergy among the combination of some compounds (antioxidants in preventive medicine. (Rev. Invest. Clin. (it refers to a Clinical Research Magazine) from 2008, 60 (1) 58-67), which led to do tests with combinations of different antioxidant substances.

Although Resveratrol and Vitamin E have been already used in dogs to benefit from the antioxidant action of Polyphenols, the quantification of the damage resulting from the oxidative stress caused in the DNA has never been evaluated in dogs.

Moreover, it is widely known that animals, especially dogs, have difficulty in taking oral medication due to the low palatability that usually characterizes veterinary compositions.

The techniques employed with the aim to make these animals swallow their medications vary from putting a pill inside their mouth and keeping it closed while closing their nose, and so forcing them to swallow the pill, to hiding a pill inside a piece of food.

These techniques can be very aggressive and complicated depending on the mood and type of animal, and it may require a lot of time to fulfill the objective.

Therefore, on one hand, it is necessary to have veterinary compositions of excellent palatability in order to avoid the rejection from these animals, and thus facilitate their treatment.

On the other hand, it is necessary to have a dietary complement composition that allows a combined decrease of sarcopenia, osteopenia and DNA damage from oxidative stress by employing effective antioxidants.

### SUMMARY OF THE INVENTION

In this regard, the object of this invention is a veterinary thick gel composition of high palatability, which contains antioxidants to decrease the sarcopenia, osteopenia and DNA damage from oxidative stress in dogs, which preferably includes the combination of at least two antioxidants selected from: Resveratrol, Pterostilbene, Vitamin E. Beta-Carotene, Myricetin and Quercetin at a concentration between 0.1% and 5.0% w/w, which are dissolved in a carrier of high palatability together with excipients accepted for veterinary purposes.

Preferably, Vitamin E can be found in the composition as DL-alpha tocopherol acetate.

Also preferably, the used carrier of high palatability is at a concentration that varies between 0.5% and 99% w/w, and it is selected from: hydrolyzed poultry liver, chicken fat, palatable agent based on bovine offal (D'Tech 12L 10975) and their mixtures, and to which vegetable oil, meat meal, cornflour, xanthan gum, whole wheat, beet pulp, brewer's yeast, corn starch, and their mixtures are additionally added.

More preferably, the veterinary thick gel composition includes between 0.3% and 0.8% w/w of Tween 80 emulsifier.

Even more preferably, the veterinary thick gel composition is divided in pouches of 5.0 g or tubes of 250 g.

In addition, there is another object of this invention: a procedure for obtaining the above mentioned veterinary thick gel composition, which consists of:
a) Placing a carrier of high palatability, the emulsifier and water in a stainless steel container;
b) Shacking with a homogenizer and adding at least one stabilizer in necessary quantities
c) Adding the selected antioxidants in necessary quantities to the mix;
d) Shacking for approximately 30 minutes until a uniform thick gel is obtained; and
e) Dividing the composition obtained in step d) in the corresponding container.

In a preferred form, the antioxidants that are added to the mix in step c) of the above procedure are selected from the combination of at least two antioxidants selected from: Resveratrol, Pterostilbene, Vitamin E, Beta-Carotene, Myricetin and Quercetin at a concentration between 0.1% and 5.0% w/w.

Preferably, Vitamin E can be found in the veterinary thick gel composition obtained from the above procedure as DL-alpha tocopherol acetate.

Also preferably, the carrier of high palatability, which is used in the procedure, is at a concentration that varies between 0.5% and 99% w/w, and it is selected from: hydrolyzed poultry liver, chicken fat, palatable agent based on bovine offal (D'Tech 12L 10975) and their mixtures, and to which vegetable oil, meat meal, cornflour, xanthan gum, whole wheat, beet pulp, brewer's yeast, corn starch, and their mixtures are additionally added.

More preferably, the veterinary thick gel composition, which is obtained from the above procedure, includes between 0.3% and 0.8% w/w of Tween 80 emulsifier.

Even more preferably, the veterinary thick gel composition, which is obtained from the above procedure, is divided in pouches of 5.0 g or tubes of 250 g.

### BRIEF DESCRIPTION OF THE DRAWINGS

FIG. 1 shows a graphic of the evolution of the total amount of serum Alkaline Phosphatase during 45 days of treatment with the veterinary thick gel composition described in Example 1 according to a preferred embodiment of the invention, and 45 days later without treatment.

FIG. 2 shows a graphic of the evolution of Creatine Phosphokinase (CPK) during 45 days of treatment with the veterinary thick gel composition described in Example 1 according to a preferred embodiment of the invention, and 45 days later without treatment.

FIG. 3 shows a graphic of the evolution of 8-Hydroxydeoxyguanosine (8(OH)dG) during 45 days of treatment with the veterinary thick gel composition described in Example 1 according to a preferred embodiment of the invention.

### DETAILED OF THE DESCRIPTION

In this regard, and with the aim to solve the problems already existing in prior art, it is hereby proposed a veterinary thick gel composition of high palatability for dogs, and which includes antioxidants capable of decreasing in a combined form the sarcopenia, osteopenia and DNA damage from oxidative stress in said animals. At the same time, it would enlarge and improve their quality of life.

For this purpose, a preferable form to put this invention into practice is through a veterinary thick gel composition of high palatability that includes antioxidants.

Therefore, it is an object of this invention a veterinary thick gel antioxidant composition of high palatability that contains antioxidants to decrease sarcopenia, osteopenia and DNA damage from oxidative stress in dogs. Preferably, it contains the combination of at least two antioxidants selected from: Resveratrol, Pterostilbene, Vitamin E, Beta-Carotene, Myricetin and Quercetin at a concentration between 0.1% and 5.0% w/w, which are dissolved in a carrier of high palatability together with excipients accepted for veterinary purposes.

Preferably, Vitamin E can be found in the veterinary thick gel composition as DL-alpha tocopherol acetate.

In this particular case, a carrier of high palatability is used at a concentration that varies between 0.5% and 99% w/w. This carrier consists of the following components selected from: hydrolyzed poultry liver, chicken fat, palatable agent based on bovine offal (D'Tech 12L 10975), and their mixtures. It is possible to add to these components vegetable oil, meat meal, cornflour, xanthan gum, whole wheat, beet pulp, brewer's yeast, corn starch, and their mixtures. In this way, it is obtained a carrier that is widely and tamely accepted by the treated animals; therefore, avoiding the problems related to the ingestion of the veterinary thick gel composition with antioxidants of this invention.

Additionally, the veterinary thick gel composition of the invention includes between 0.3% and 0.8% w/w of Tween 80 emulsifier.

In a preferred form, the veterinary thick gel composition of the invention is divided in pouches of 5.0 g or tubes of 250 g for its commercialization.

In addition, there is another object of this invention: a procedure for obtaining the veterinary thick gel composition previously described, which consists of:
a) Placing a carrier of high palatability, the emulsifier and water in a stainless steel container;
b) Shacking with a homogenizer and adding at least one stabilizer in necessary quantities;
c) Adding the selected antioxidants in necessary quantities to the mix;
d) Shacking for approximately 30 minutes until a uniform thick gel is obtained; and
e) Dividing the composition obtained in step d) in the corresponding container.

The antioxidants that are added to the mix in step c) are selected from the combination of at least two antioxidants selected from: Resveratrol. Pterostilbene. Vitamin E, Beta-Carotene, Myricetin and Quercetin at a concentration between 0.1% and 5.0% w/w.

Particularly, the Vitamin E that is added in step c) can be found as DL-alpha tocopherol acetate.

The carrier of high palatability used in step a) from the above procedure is added at a concentration that varies between 0.5% and 99% w/w, and it is selected from: hydrolyzed poultry liver, chicken fat, palatable agent based on bovine offal (D'Tech 12L 10975), and their mixtures, and to which vegetable oil, meat meal, corn flour, xanthan gum, whole wheat, beet pulp, brewer's yeast, corn starch, and their mixtures are additionally added.

Furthermore, the procedure of this invention preferably consists of the addition between 0.3% and 0.8% w/w of Tween 80 emulsifier in step a).

In a preferential form, the way of dividing the veterinary thick gel composition of step e) is in pouches of 5.0 g or tubes of 250 g.

### Example 1

The following veterinary composition in a thick antioxidant gel of high palatability has been prepared, and it has been administered to a group of dogs, which have thoroughly accepted it without rejection.

Veterinary Composition in a Thick Antioxidant Gel 1

| | Compo- | Quantity (mg) | Quantity (mg) |
|---|---|---|---|
| | sition | Per tube | Per pouch |
| Components | (%) | of 250 g | of 5 g |
| Hydrolyzed Poultry Liver | 54.15 | 135,300 | 2,706 |
| Resveratrol | 0.60 | 1500 | 30 |
| Vitamin E as DL-alpha | 0.80 | 2000 (Equivalent | 40 (Equivalent |
| tocopherol acetate 50% | | to 10 IU) | to 20 IU) |
| Chicken Fat | 38.36 | 96,000 | 1,920 |
| Water | 5.4 | 13,500 | 270 |
| Tween 80 | 0.56 | 1400 | 28 |
| Nipagin sodium | 0.108 | 270 | 5.4 |
| Nipasol sodium | 0.028 | 70 | 1.4 |

### Example 2

The following veterinary composition in a thick antioxidant gel of high palatability has been prepared, and it has been administered to a group of dogs, which have thoroughly accepted it without rejection.

Veterinary Composition in a Thick Antioxidant Gel 2

| | Compo- | Quantity (mg) | Quantity (mg) |
|---|---|---|---|
| | sition | Per tube | Per pouch |
| Components | (%) | of 250 g | of 5 g |
| Hydrolyzed Poultry Liver | 54.15 | 135,300 | 2,706 |
| Resveratrol | 0.60 | 1500 | 30 |
| Pterostilbene | 0.80 | 2000 | 40 |
| Corn Oil | 38.36 | 96,000 | 1,920 |
| Water | 5.4 | 13,500 | 270 |
| Tween 80 | 0.56 | 1400 | 28 |
| BHT | 0.108 | 270 | 5.4 |
| BHA | 0.028 | 70 | 1.4 |

### Example 3

The following veterinary composition in a thick antioxidant gel of high palatability has been prepared, and it has been administered to a group of dogs, which have thoroughly accepted it without rejection.

Veterinary Composition in a Thick Antioxidant Gel 3

| | Compo- | Quantity (mg) | Quantity (mg) |
|---|---|---|---|
| | sition | Per tube | Per pouch |
| Components | (%) | of 250 g | of 5 g |
| Hydrolyzed Poultry Liver | 54.15 | 135,300 | 2,706 |
| Pterostilbene | 0.60 | 1500 | 30 |
| Vitamin E as DL-alpha | 0.80 | 2000 (Equivalent | 40 (Equivalent |
| tocopherol acetate 50% | | to 10 IU) | to 20 IU) |
| Corn Oil | 38.36 | 96,000 | 1,920 |
| Water | 5.4 | 13,500 | 270 |
| Tween 80 | 0.56 | 1400 | 28 |
| Nipagin sodium | 0.108 | 270 | 5.4 |
| Potassium Sorbate | 0.028 | 70 | 1.4 |

### Example 4

The following veterinary composition in a thick antioxidant gel of high palatability has been prepared, and it has been administered to a group of dogs, which have thoroughly accepted it without rejection.

Veterinary Composition in a Thick Antioxidant Gel 4

| | Compo- | Quantity (mg) | Quantity (mg) |
|---|---|---|---|
| | sition | Per tube | Per pouch |
| Components | (%) | of 250 g | of 5 g |
| D'Tech 12L 10975 | 54.15 | 135,300 | 2,706 |
| Palatable agent | | | |
| (Diana Pet Feed) | | | |
| Resveratrol | 0.60 | 1500 | 30 |
| Vitamin E as DL-alpha | 0.80 | 2000 (Equivalent | 40 (Equivalent |
| tocopherol acetate 50% | | to 10 IU) | to 20 IU) |
| Xanthan Gum | 38.36 | 96,000 | 1,920 |
| Water | 5.4 | 13,500 | 270 |
| Tween 80 | 0.56 | 1400 | 28 |
| Nipagin sodium | 0.108 | 270 | 5.4 |
| Potassium Sorbate | 0.028 | 70 | 1.4 |

### Example 5

The following veterinary composition in a thick antioxidant gel of high palatability has been prepared, and it has been administered to a group of dogs, which have thoroughly accepted it without rejection.

Veterinary Composition in a Thick Antioxidant Gel 5

| | Compo- | Quantity (mg) | Quantity (mg) |
|---|---|---|---|
| | sition | Per tube | Per pouch |
| Components | (%) | of 250 g | of 5 g |
| Hydrolyzed Poultry Liver | 54.15 | 135,300 | 2,706 |
| Vitamin E as DL-alpha | 1.0 | 5000 (Equivalent | 100 (Equivalent |
| tocopherol acetate 50% | | to 10 IU) | to 20 IU) |
| Chicken Fat | 38.36 | 96,000 | 1,920 |
| Water | 5.4 | 13,500 | 270 |
| Tween 80 | 0.56 | 1400 | 28 |
| Nipagin sodium | 0.108 | 270 | 5.4 |
| Nipasol sodium | 0.028 | 70 | 1.4 |

### Example 6

### Methodology for Preparing the Product

In order to prepare the composition of Examples 1, 2, 3, 4 and 5 the following steps were taken: the carriers and adjuvants (fats, hydrolisates, emulsifiers, etc.) were placed in a stainless steel container. It was shaked with a homogenizer, and the preservatives were added in necessary quantities. Then the antioxidants were added in the required quantities, and it was shaked for about 30 minutes until obtaining a uniform consistency. Finally, it was divided in the corresponding containers.

Particularly, the chicken fat that is used in the preparation of a veterinary thick gel composition, which is object of this invention, is a stabilized product resulting from the cooking process of fresh chickens, and obtained from adipose tissue. The ingredients that comprise this fat are 99,97% of chicken fat, 0.01% of BHA, 0.001% of propyl gallate and 0.001% of citric acid.

The process for obtaining stabilized poultry fat, for example chicken fat, includes the following: a) cooking the chickens; b) pouring; c) removing the chicken fat; d) pouring; e) adding antioxidant and shaking; and f) packing it at a 70° C. temperature in buckets containing polyethilene bags in their interior for its closure and commercialization.

The typical veterinary thick gel composition per each 100 g of chicken fat is the following: total fat: 99.7 g; saturated fats: 22 g; monounsaturated fats: 40 g; polyunsaturated fats: 37 g, and trans fats: 0.15 g; with an energy value of 3,772 kJ (898 kcal).

Regarding the hydrolyzed powdery liver, it is obtained by an enzimatic hydrolysis of poultry livers, for example chicken, which result thereof is then dried by the spray drying method. The ingredients of this raw material, which is identified as poultry liver, includes amino acids and nuclides. BHA, and carbohydrates. It is powdery, light brown, with a characteristic smell and flavour.

The typical analysis of this product when it is chicken liver, is as follows: maximum humidity of 8%; fat from 13% to 18%, protein in the order of 52%; a peroxide value of 2 meq/kg; BHA qualitative antioxidants; Propyl Gallate and citric acid; and ashes in the order of 8%.

The palatable agent D'Tech 12L 10975, which is a product from the company Diana Pet Food, is a high-palatability liquid enhancer with a protein content bigger or equal to 10% and with a fat content lower or equal to 6%. This product is prepared upon bovine offal and preservatives such as Phosphoric acid. Potassium Sorbate, Ascorbic acid, citric acid, Butylated Hydroxyanisole and Propilgalate.

### Example 7

### Performance and Application of the Compositions in Thick Antioxidant Gels of the Invention

The oxidative metabolism is the main responsible for generating metabolic waste and debris in mitochondria when producing more than 95% of free radicals. From 3% to 5% of metabolic debris are not eliminated, and this accumulation of free radicals is one of the main causes for noncommunicable chronic diseases, such as obesity, diabetes, cardiovascular, kidney, bone and joints, neurodegenerative and oncologic diseases. When the free radicals exceed the antioxidant capacity of the cell, the so-called oxidative stress is produced. Aiming to decrease the cell damages produced by the free radicals, which are determinant for the beginning of the mentioned diseases, the treatment was evaluated with combinations of different antioxidants according to Examples 1, 2, 3, 4 and 5 as a dietary complement. Its effects on the biology were measured with serum biomarkers of oxidative damage to the DNA, muscles and bones.

### Used Product

Thick gel compositions, which were obtained from Examples 1, 2, 3, 4 and 5 based on raw materials approved for their use in veterinary products, were used.

### Materials and Methods

Studies were carried out with samples of each one of the Examples 1, 2, 3, 4 and 5, and for each study in each case it was selected a population of 22 male and female, neutered dogs, which were adults of different ages, from indefinite breed, with no diseases shown in clinical and biochemical tests, without parasites, with no medication or dietary complements being administered to them, and treated with equal hygienic and food conditions. In day 0 (D0) a basal clinical test with blood taking was made to determine the biomarkers. During 45 days all the dogs were administered with 2 daily doses of 5 g of the product, each one with each main meal. When the treatment stage ended, in day 45 (D45) a new clinical test and blood taking were made. As from day 45 and until day 90 (D90), all the dogs were kept under the same general conditions, but without the daily provision of dietary complement. In D90 new blood tests were taken for the analysis of biomarkers. All the serums were kept in a freezer at −80° C. until the day of their analysis. The evaluated biomarkers were: 8-Hydroxydeoxyguanosine (8(OH)dG), (ELISA method), Creatine Phosphokinase (CPK) and total and bone Alkaline Phosphatase (ALP) (spectrophotometry and immunoenzymatic assay methods). The statistic analysis of D0 vs D45 and D45 vs D90 were made by the Student's-T Test for paired samples.

TABLE 1
The results were obtained by using the veterinary
composition in thick antioxidant gel of Example 1

| | Day 0 | Day 45 | p- | Day 45 | Day 90 | p- |
|---|---|---|---|---|---|---|
| Biomarker | (D 0) | (D 45) | value | (D 45) | (D 90) | value |
| 8-Hydroxy- | 3,266.7 ± 1,966.1 | 2,856.1 ± 1,380.6 | −13% | | | |
| deoxyguanosine | | | | | | |
| (8(OH)dG) | | | | | | |
| (pg/ml) | | | | | | |
| Creatine | 252 ± 107.0 | 176.5 ± 69.4 | <0.001 | 176.5 ± 69.4 | 218.4 ± 67.9 | <0.01 |
| Phosphokinase | | | | | | |
| CPK (IU/L) | | | | | | |
| Alkaline | 99.3 ± 43.0 | 76.3 ± 27.3 | <0.001 | 76.3 ± 27.3 | 140.7 ± 93.7 | <0.001 |
| Phosphatase | | | | | | |
| (ALP) (IU/L) | | | | | | |

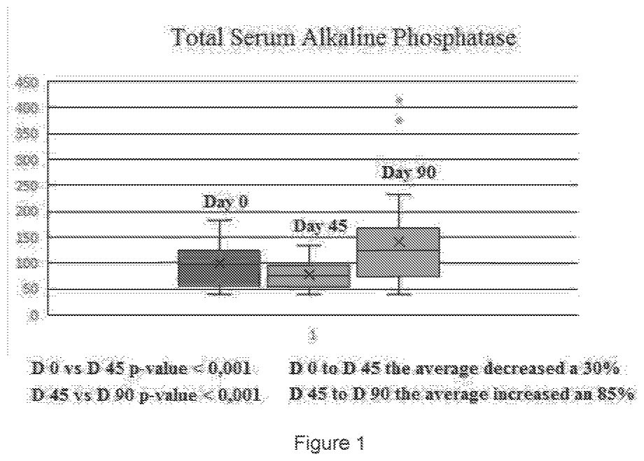

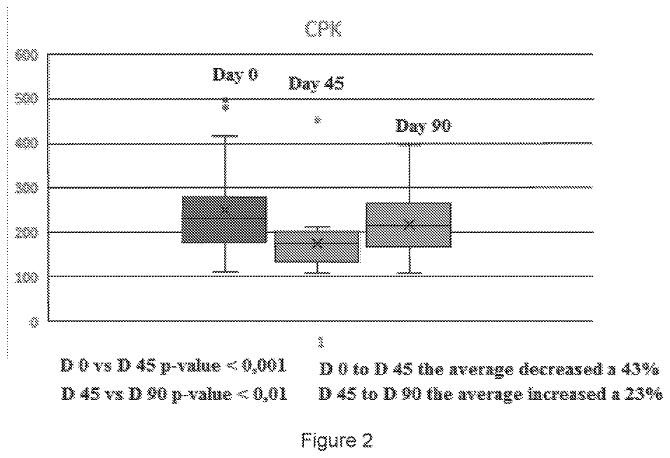

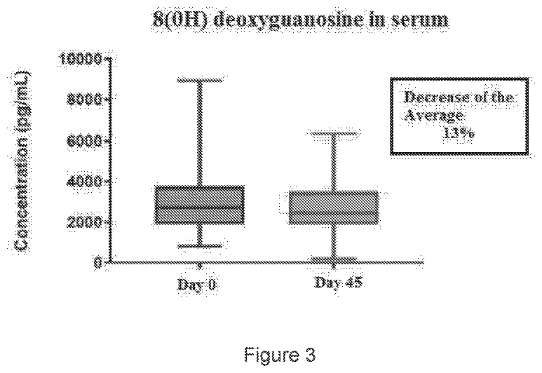

Please refer to graphics from FIGS. 1, 2 and 3.

TABLE 2
The results were obtained by using the veterinary
composition in thick antioxidant gel of Example 2

| | Day 0 | Day 45 | p- | Day 45 | Day 90 | p- |
|---|---|---|---|---|---|---|
| Biomarker | (D 0) | (D 45) | value | (D 45) | (D 90) | value |
| 8-Hydroxy- | 3,293.5 ± 1,978.1 | 2,776.1 ± 1,380.6 | −15% | | | |
| deoxyguanosine | | | | | | |
| (8(OH)dG) | | | | | | |
| (pg/ml) | | | | | | |
| Creatine | 250 ± 103.0 | 166.8 ± 69.4 | <0.001 | 166.8 ± 69.4 | 208.7 ± 66.5 | <0.01 |
| Phosphokinase | | | | | | |
| CPK (IU/L) | | | | | | |
| Alkaline | 90.5 ± 40.2 | 73.3 ± 25.2 | <0.001 | 73.3 ± 25.2 | 114.9 ± 95.2 | <0.001 |
| Phosphatase | | | | | | |
| (ALP) (IU/L) | | | | | | |

TABLE 3
The results were obtained by using the veterinary
composition in thick antioxidant gel of Example 3

| | Day 0 | Day 45 | p- | Day 45 | Day 90 | p- |
|---|---|---|---|---|---|---|
| Biomarker | (D 0) | (D 45) | value | (D 45) | (D 90) | value |
| 8-Hydroxy- | 3,138.5 ± 1,746.0 | 2,856.1 ± 1,380.6 | −12% | | | |
| deoxyguanosine | | | | | | |
| (8(OH)dG) | | | | | | |
| (pg/ml) | | | | | | |
| Creatine | 243 ± 97.0 | 163.5 ± 65.3 | <0.001 | 163.5 ± 65.3 | 209.4 ± 59.9 | <0.01 |
| Phosphokinase | | | | | | |
| CPK (IU/L) | | | | | | |
| Alkaline | 89.5 ± 39.0 | 78.5 ± 19.8 | <0.001 | 78.5 ± 19.8 | 110.5 ± 96.3 | <0.001 |
| Phosphatase | | | | | | |
| (ALP) (IU/L) | | | | | | |

TABLE 4
The results were obtained by using the veterinary
composition in thick antioxidant gel of Example 4

| | Day 0 | Day 45 | p- | Day 45 | Day 90 | p- |
|---|---|---|---|---|---|---|
| Biomarker | (D 0) | (D 45) | value | (D 45) | (D 90) | value |
| 8-Hydroxy- | 3,259.8 ± 1,899.3 | 2,856.1 ± 1,380.6 | −14% | | | |
| deoxyguanosine | | | | | | |
| (8(OH)dG) | | | | | | |
| (pg/ml) | | | | | | |
| Creatine | 256 ± 110.0 | 183.5 ± 69.9 | <0.001 | 183.5 ± 69.9 | 227.4 ± 67.0 | <0.01 |
| Phosphokinase | | | | | | |
| CPK (IU/L) | | | | | | |
| Alkaline | 98.9 ± 43.0 | 76.0 ± 25.4 | <0.001 | 76.0 ± 25.4 | 100.7 ± 39.5 | <0.001 |
| Phosphatase | | | | | | |
| (ALP) (IU/L) | | | | | | |

TABLE 5
The results were obtained by using the veterinary
composition in thick antioxidant gel of Example 5

| | Day 0 | Day 45 | p- | Day 45 | Day 90 | p- |
|---|---|---|---|---|---|---|
| Biomarker | (D 0) | (D 45) | value | (D 45) | (D 90) | value |
| 8-Hydroxy- | 3,266.7 ± 1,873.1 | 3,171.5 ± 1,255.6 | −3% | | | |
| deoxyguanosine | | | | | | |
| (8(OH)dG) | | | | | | |
| (pg/ml) | | | | | | |
| Creatine | 252 ± 107.0 | 246.5 ± 66.5 | <0.001 | 246.5 ± 66.5 | 250.4 ± 67.9 | <0.01 |
| Phosphokinase | | | | | | |
| CPK (IU/L) | | | | | | |
| Alkaline | 99.3 ± 43.0 | 96.9 ± 27.3 | <0.001 | 96.9 ± 27.3 | 98.7 ± 33.7 | <0.001 |
| Phosphatase | | | | | | |
| (ALP) (IU/L) | | | | | | |

### CONCLUSIONS

The evaluated biomarkers let us assure that the provision of antioxidants, particularly the mixtures of at least two antioxidants selected from Resveratrol. Pterostilbene and Vitamin E, as a dietary complement had the following effects. 1) decrease of oxidative stress, 2) highly significant decrease in the loss of skeletal muscle (CPK), 3) highly significant decrease in the loss of bone mass (ALP), and 4) decrease of 12-15% in the loss of (8(OH)dG) DNA. The significant increase in the values of biomarkers as of D90 shows that the benefit obtained from the combination of Resveratrol and other antioxidant as of D45, is lost when removing it from the daily diet. It is the first time that it is demonstrated that a dietary complement provided to healthy dogs decreases the loss of muscle mass, bone mass and DNA from oxidative stress. It is also possible to observe that the Vitamin E that was provided alone produced a lesser effect, compared to that of the mixture of two antioxidants that were jointly provided.

1. A veterinary thick gel composition of high palatability containing antioxidants to decrease sarcopenia, osteopenia and DNA damage from oxidative stress in dogs, comprising the combination of at least two antioxidants selected from: Resveratrol, Pterostilbene, Vitamin E, Beta-Carotene, Myricetin and Quercetin at a concentration between 0.1% and 5.0% w/w, which are dissolved in a carrier of high palatability together with excipients accepted for veterinary purposes.

2. The veterinary thick gel composition of claim 1, wherein Vitamin E is in the form of DL-alpha tocopherol acetate.

3. The veterinary thick gel composition of claim 1 wherein the carrier of high palatability is at a concentration that varies between 0.5% and 99% w/w, and is selected from: hydrolyzed poultry liver, chicken fat, palatable agent based on bovine offal (D'Tech 12L 10975) and their mixtures, and to which vegetable oil, meat meal, corn flour, xanthan gum, whole wheat, beet pulp, brewer's yeast, corn starch, and their mixtures are additionally added.

4. The veterinary thick gel composition of claim 1, which comprises between 0.3% and 0.8% w/w of Tween 80 emulsifier.

5. The veterinary thick gel composition of claim 1 which is divided in pouches of 5.0 g or in tubes of 250 g.

6. A procedure to obtain the veterinary thick gel composition of claim 1 comprises:
Placing a carrier of high palatability, the emulsifier and water in a stainless steel container;
Shacking with a homogenizer and adding at least one stabilizer in necessary quantities;
Adding to the mix the selected antioxidants in necessary quantities;
Shacking for approximately 30 minutes until a uniform thick gel is obtained; and
Dividing the composition obtained in step d) in the corresponding container.

7. The procedure of claim 6, wherein the antioxidants that are added to the mixture in step c) are selected from the combination of at least two antioxidants selected from: Resveratrol, Pterostilbene, Vitamin E, Beta-Carotene, Myricetin and Quercetin at a concentration between 0.1% and 5.0% w/w.

8. The procedure of claim 6, wherein Vitamin E is in the form of DL-alpha tocopherol acetate.

9. The procedure of wherein the carrier of high palatability is at a concentration that varies between 0.5% and 99% w/w, and it is selected from: hydrolyzed poultry liver, chicken fat, palatable agent based on bovine offal (D'Tech 12L 10975) and their mixtures, and to which vegetable oil, meat meal, corn flour, xanthan gum, whole wheat, beet pulp, brewer's yeast, corn starch, and their mixtures are additionally added.

10. The procedure of claim 6, which comprises between 0.3% and 0.8% w/w of Tween 80 emulsifier.

11. The procedure of claim 6, wherein the veterinary thick gel composition is divided in pouches of 5.0 g or in tubes of 250 g.

12. The veterinary thick gel composition of claim 2, wherein the carrier of high palatability is at a concentration that varies between 0.5% and 99% w/w, and is selected from: hydrolyzed poultry liver, chicken fat, palatable agent based on bovine offal (D'Tech 12L 10975) and their mixtures, and to which vegetable oil, meat meal, corn flour, xanthan gum, whole wheat, beet pulp, brewer's yeast, corn starch, and their mixtures are additionally added.

13. The veterinary thick gel composition of claim 2, which comprises between 0.3% and 0.8% w/w of Tween 80 emulsifier.

14. The veterinary thick gel composition of claim 3, which comprises between 0.3% and 0.8% w/w of Tween 80 emulsifier.
